(19) United States
(12) Plant Patent Application Publication
(10) Pub. No.: US 2004/0045062 P1
(43) Pub. Date: Mar. 4, 2004

(54) Sugar maple named "Hawkersmith 1"

(76) Inventor: Louis A. Hawkersmith, Tullahoma, TN (US)

Correspondence Address: William G. Bruns, Polster, Lieder, Woodruff & Lucchesi, L.C., 763 South New Ballas, St. Louis, MO, 63141, US

(21) Appl. No.: 10/230,453

(22) Filed: Aug. 29, 2002

(51) Int. Cl. A01H 5/00

(52) U.S. Cl. PLT/224

(57) ABSTRACT

A new culivar of Sugar Maple tree (Acer saccharum) having a compact oval appearance, dark red to bronze new growth and fall foliage, small, dark and slightly thicker leaves, good summer heat and drought resistance and cold hardiness. The tree is beautiful, compact, tought, colorful and functional as an ornamental shade tree.

### CROSS REFERENCE TO RELATED APPLICATIONS

### STATEMENT REGARDING FEDERALLY SPONSORED RESEARCH OR DEVELOPMENT

### BACKGROUND OF THE INVENTION

The present invention relates to a new and distinct variety of Sugar Maple tree which I have named “HAWKERSMITH 1”.

The original Sugar Maple tree of the new cultivar was selected in 1985 from a block of seedlings planted in rows in a production field on the property of Hawkersmith and Sons Nursery of Tullahoma, Tenn. The seedlings were planted in rows and designed to be sold at a caliper of 4-10 inches. The selection was made on the basis of the distinctive characteristics of a single tree (as hereinafter described) noted after a few years of growth. The new cultivar was growing in a seeding block of standard Acer saccharum trees. The seed source is unknown and the origin of the seedlings is unknown.

The new cultivar has been asexually reproduced successfully by chip budding onto Acer saccharum rootstock at Heartland Nursery Company at New Madrid, Mo.

It has been found that the new Acer saccharum cultivar exhibits the following combination of characteristics.

1. A much smaller leaf than other Sugar Maples.

2. A slightly darker leaf than other Sugar Maples.

3. A slightly thicker leaf than other Sugar Maples based on feel and appearance, although the precise thickness has not been measured.

4. In full leaf, the branching structure has a layered appearance.

5. When dormant and in full leaf, the Sugar Maple looks as if it has been sheared (very dense branching).

6. The new growth is dark red to bronze.

7. The fall color is very similar to new growth.

8. The tree is almost semi-dwarf.

9. The rate of growth is-moderate when young and very slow after 10 years.

10. The bark is gray.

11. The tree is very tolerant to summer heat of the south.

12. The tree has no winter die back and is very cold hardy.

13. The color of new growth and fall color seem to be consistent from year to year.

14. The leaves turn fall color 2-3 weeks later than other Sugrar Maples.

The tree characteristics following asexual reproduction are the same as those of the original tree of the new cultivar. The tree has shown no winter die back and is cold hardy. It also is very tolerant of southern summer heat and is more drought tolerant compared to other Sugar Maples grown under the same circumstances.

The new cultivar of the present invention has been named the “HAWKERSMITH 1” cultivar and is being marketed under the AUTUMN FAITH trademark.

### DESCRIPTION OF THE PHOTOGRAPHS

The accompanying photographs show, as nearly true as is reasonably possible to make the same in color illustrations of this character, typical specimens and plant parts of the new cultivar. The tree depicted in the photograph is growing in Tullahoma, Tenn.

FIG. 1 illustrates during the month of July a mature tree of the new cultivar at an age of approximately 20 years. The trunk diameter is about 8 inches, the height is about 25 feet and the maximum breath is approximately 15 feet.

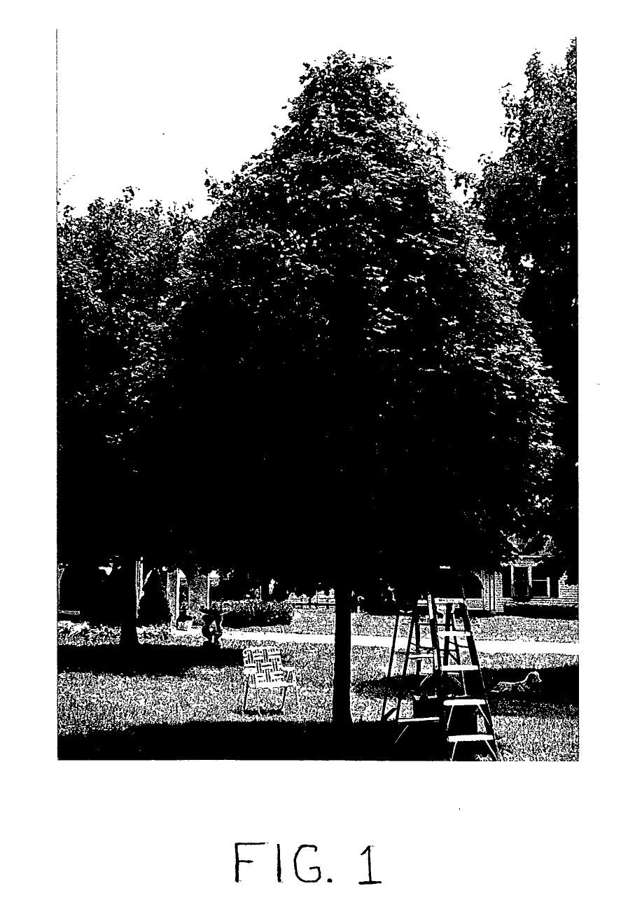

FIG. 2 illustrates the tree of FIG. 1 during the month of November. Fall coloration in shades of dark red and bronze is apparent.

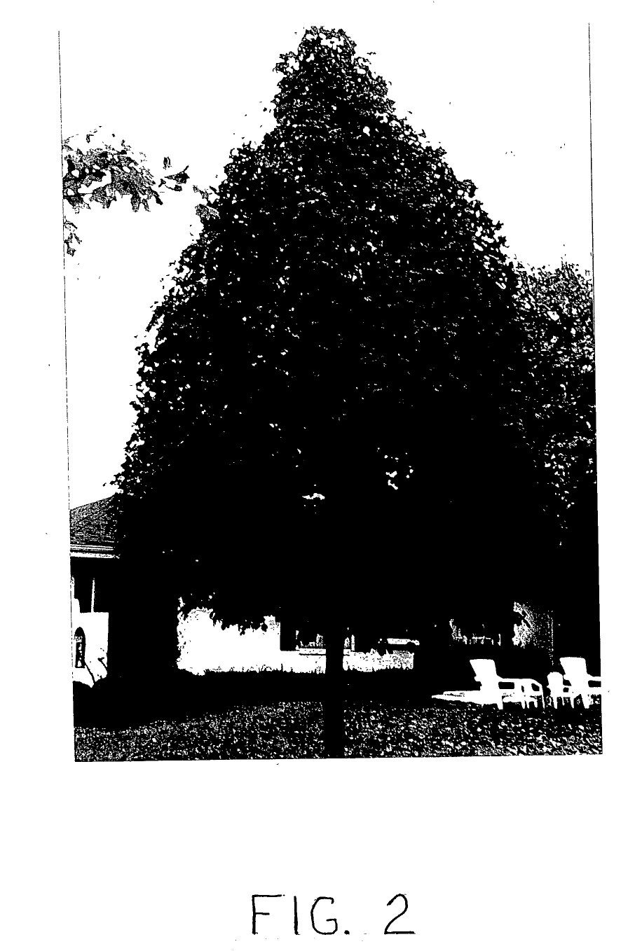

FIG. 3 shows a close-up of the dense summer foliage of the tree of FIG. 1.

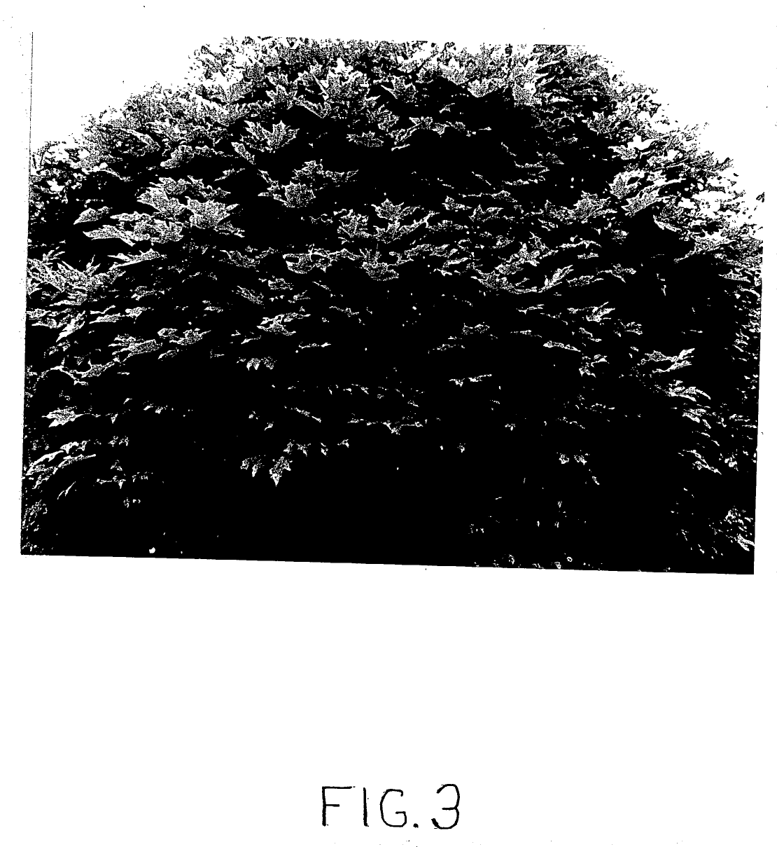

FIG. 4 is a close-up of individual leaves showing the smaller darker mature leaf of this tree.

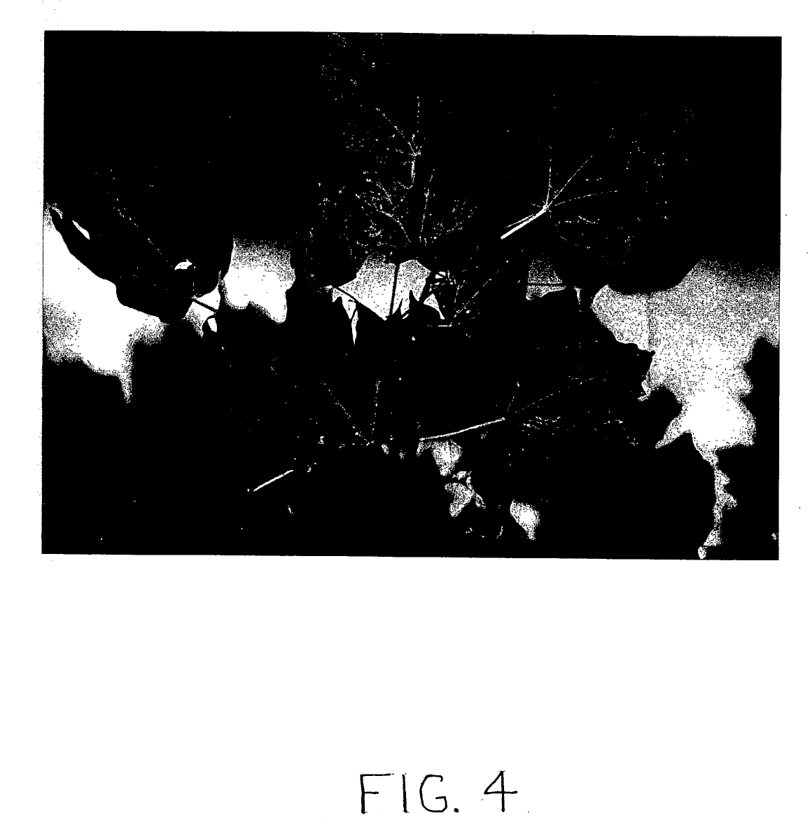

FIG. 5 shows the new leaf growth of the tree during the month of May in shades of dark red and bronze.

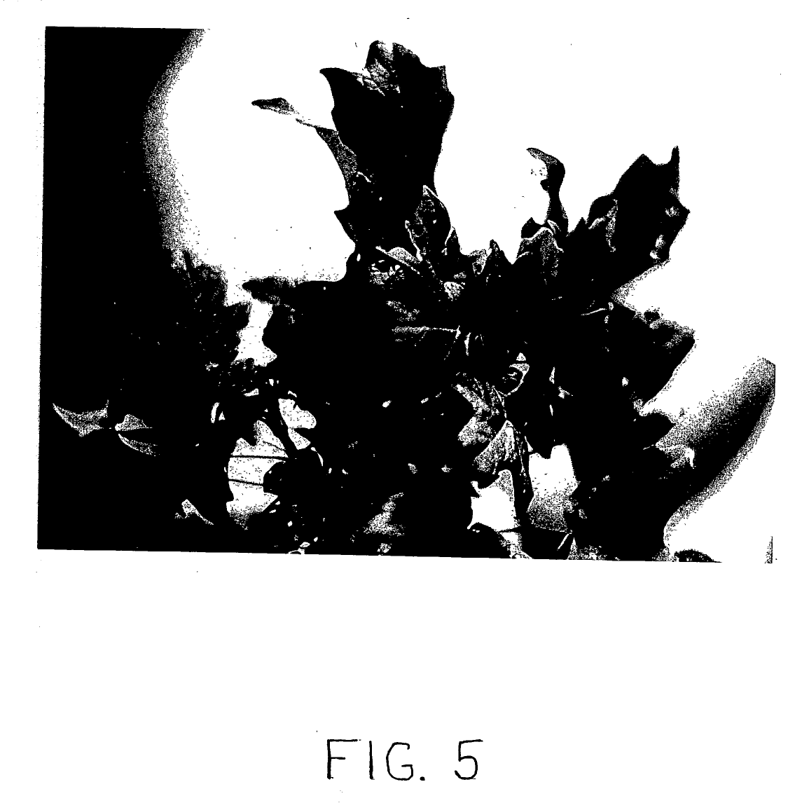

FIG. 6 shows the gray bark and the bud sets varying from ¼ inch at the ends of the branch to 2 inches apart toward the center of the tree.

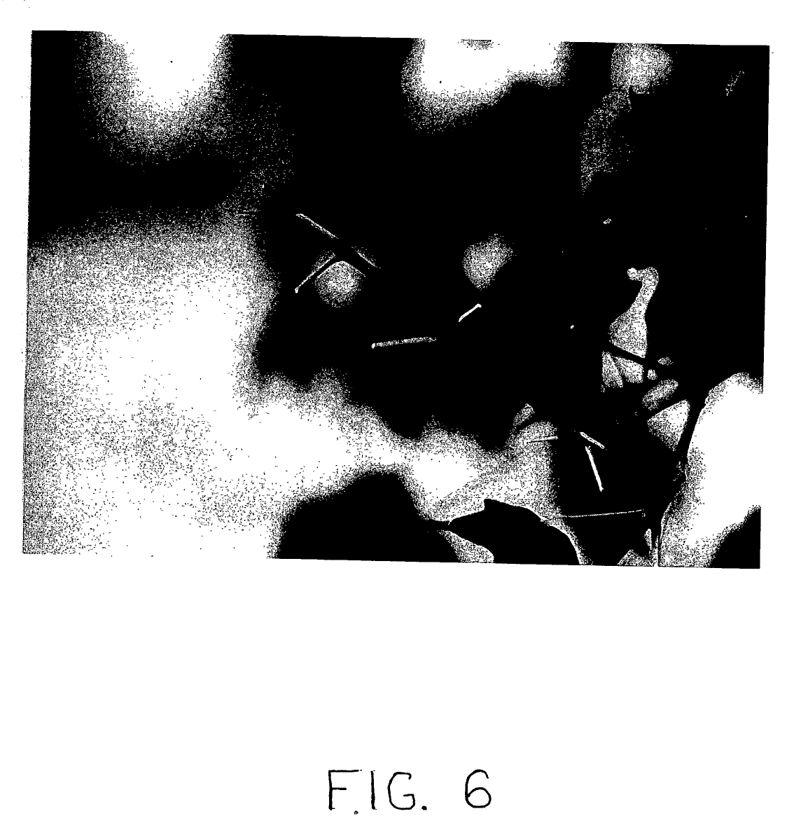

### DETAILED DESCRIPTION

The following is a detailed description of my new variety of Sugar Maple. The chart used in identification of colors is that of The Royal Horticulture Society (R.H.S. Colour Chart). Common color terms are to be accorded their ordinary dictionary meaning. The description is based on a test planting of the new variety at New Madrid, Mo.

Parentage: Chance seeding believed to be of Acer saccharum of unknown origin found growing in a cultivated area in Tullahoma, Tenn.

Tree Shape: Upright, oval with layered very dense branch structure.

Size: Smaller than standard Acer saccharum of same age; almost semi-dwarf. The mature size should be no more than 35 feet.

Growth: Moderate when young; very slow after 10 years.

Trunk: Generally straight and upright.

Bark: Gray.

Leaves: Much smaller, slightly darker and slightly thicker than standard Sugar Maple.

Color: The new growth and fall color is dark red to bronze. The summer color is darker green than standard Acer saccharum. The leaves turn their fall color 2-3 weeks later than standard Sugar Maples.

Bud sets: Vary from ¼″ apart at the ends of the branches to about 2″ apart toward the trunk.

Reproductive organs and fruit: Typical of those of standard Sugar Maple.

Ability to withstand head: The tree is tolerant of summer heat in the South and is very drought tolerant compared to standard Sugar Maple.

Cold hardiness: The tree has no winter die back. It also has withstood high winds and ice.

Disease Resistance: No disease or insect problems have been noted.

Leaf color comparison with The Royal Horticultural Society Colour Chart:

New stem.—Fan 3. Yellow-Green group. 146 B.

New leaf.—Fan 4. Greyed-Purple group. 185 B.

Leaf stem.—Fan 3. Yellow-Green group. N 144 D.

Summer leaf.—Fan 3. Yellow-Green group. 147 A.

Old stem.—Fan 4. Greyed-Green group. 197 C.

Fall leaf.—Fan 4. Greyed-Purple group 184 C.

What is claimed is:

1. A new and distinct variety of Sugar Maple tree substantially as herein shown and described, characterized particularly as to novelty by a compact shape, moderate to slow growth, small, dark and somewhat thicker leaves, good drought and summer heat resistance, resistance to wind and cold, and new growth and fall coloration being dark red to bronze.
